(19) United States
(12) Patent Application Publication
(10) Pub. No.: US 2019/0290456 A1
(43) Pub. Date: Sep. 26, 2019

(54) ELECTROACTIVE POLYMERS THAT CONTRACT AND EXPAND, SENSE PRESSURE, AND ATTENUATE FORCE AND SYSTEMS USING THE SAME

(71) Applicant: RAS Labs, Inc., Quincy, MA (US)

(72) Inventors: Lenore RASMUSSEN, Hingham, MA (US); Leila ALBERS, Braintree, MA (US); Simone RODRIGUEZ, Williamstown, MA (US)

(21) Appl. No.: 16/307,225

(22) PCT Filed: Jun. 23, 2017
(86) PCT No.: PCT/US2017/038968

§ 371 Date: Dec. 5, 2018

Related U.S. Application Data
(60) Provisional application No. 62/353,806, filed on Jun. 23, 2016.
(60) Provisional application No. 62/405,222, filed on Oct. 6, 2016.
(60) Provisional application No. 62/431,804, filed on Dec. 8, 2016.
(60) Provisional application No. 62/433,447, filed on Dec. 13, 2016.
(60) Provisional application No. 62/484,848, filed on Apr. 12, 2017.
(60) Provisional application No. 62/519,820, filed on Jun. 14, 2017.

(51) Int. Cl. A61F 2/78; C08L 25/18; A61F 2/76; A61F 5/14; A61F 2/80

(52) U.S. Cl. CPC A61F 2/7812; C08L 25/18; A61F 2/76; C08L 33/14; A61F 2/80; C08L 2203/02; A61F 2002/7635; A61F 5/14

(57) ABSTRACT

Novel robust electroactive polymers (EAPs) and EAP-based systems are described, which contract and expand at low voltages to provide for a shape-morphing system, which also sense mechanical pressure, from gentle touch to high impact, and which attenuate force. These EAPs and EAP-based systems can be used in a prosthetic liner, and potentially as the entire prosthetic liner, in a prosthetic hard socket, in shoe wear, sports gear, protective gear, and military gear, and in compression equipment, to contract and expand in strategic areas as needed to maintain a perfect fit, to sense pressure and provide feedback to automatically maintain perfect fit, and to attenuate force for an extremely comfortable fit.

### INCORPORATION BY REFERENCE

All patents, patent applications, and publications cited herein are hereby incorporated by reference in their entirely in order to more fully describe the state of the art as known to those skilled therein as of the date of the invention described herein.

### RELATED APPLICATIONS

The present application claims the benefits of and priority to U.S. Provisional Application 62/353,806, filed Jun. 23, 2016, U.S. Provisional Application 62/405,222, filed Oct. 6, 2016, U.S. Provisional Application 62/431,804, filed Dec. 8, 2016, U.S. Provisional Application 62/433,447, filed Dec. 13, 2016, U.S. Provisional Application 62/484,848, filed Apr. 12, 2017, and U.S. Provisional Application 62/519,820, filed Jun. 14, 2017, the contents of which are hereby incorporated by reference herein in their entirety.

### BACKGROUND

Traditional helmet padding is composed of passive materials that attenuate impact but only change shape with applied pressure and have no role or capability to sense potentially injurious impacts. The electroactive polymers (EAPs) and EAP-based systems in the instant invention are ideally suited for shape-morphing, impact sensing, and for impact attenuation since these EAPs are neither a pure solid nor a pure liquid. Due to the EAP's semi-solid composition and viscoelastic and damping properties, the EAPs and EAP-based systems in the instant invention exhibit non-Newtonian behavior.

Traditional shoe insoles provide comfort, including therapy, for example, arch support. Some insoles also provide sensing, but no shoe insole on the market provides for shape-morphing, sensing, and force attenuation. The problem with current marketplace sensors in shoe insoles is that where the sensor is attached or is embedded in the shoe insole material causes a weak area at the interface between the sensor and the surrounding insole material, which then leads to breakdown of the insole. In the instant invention, the shape-morphing, force attenuating shoe insole material is also the sensor, which eliminates an interface between the sensor and the insole material. In addition, using the shape-morphing abilities in selected zones can provide for a perfect fitting shoe insole, providing for unsurpassed comfort and therapeutic benefit, such as perfect arch support, for example.

For prosthetic liners and pads for the hard socket, traditional liners and socket pad systems on the marketplace are fraught with problems. Most prosthetic liners and sockets are static. A mold of the residual limb is made, and then the prosthetic liner and socket are designed around the mold from the day of the amputee patient's fitting for the mold. The reality is that residual limbs are continually changing. In fact, most amputees' residual limbs shrink over the course of any given day (typical case), much like people's foot size changes from morning to evening. Current prosthetic liners use a flexible material, such as a thick layer of silicone- or polyurethane-based material, which help to hold the liner to the residual limb by its shape (fitted to patient), some suction, and the liner's elasticity. These liners are available with or without the distal locking feature and are usually worn with traditional prosthetic socks to allow for volume adjustments. As the limb shrinks over the course of a typical day, however, there often forms a significant gap between the liner and the hard shell of the socket of the prosthetic device, which can be addressed by the patient by adding more layers of cotton socks between the liner and the hard shell. If the fit becomes too tight, the patient then removes layers of cotton socks between the liner and the hard shell. This is time-consuming and cumbersome. If the patient fails to notice that the fit is becoming too loose or too tight, tissue damage can occur to the residual limb. Because the skin of the leg (below the knee amputee) and thigh (above the knee amputee) do not have many nerves compared to hands and feet, the patient often does not notice a poor fit until there is a problem, and in the case of slippage, even bleeding from abrasion, due to these areas of the body being poorly innervated. For amputees who suffer from diabetes, this problem is exacerbated.

To address the maintenance of prosthetic liner and socket fit beyond a flexible liner, several strategies have been explored, such as a variety of suction and vacuum systems. Suction systems often consist of a soft liner equipped with a one-way valve and a sealing sleeve. The patient inserts his or her liner-covered limb into the socket and the application of body weight as he or she stands expels excess air through the valve. In a typical vacuum system, a sleeve creates a seal around the top edge of the socket, then a pump and exhaust valve remove virtually all air between the socket and the liner as the patient wears the device. This system regulates the vacuum level within a defined range. Benevolent Technologies uses a pump to pull vacuum around gelled beads to produce a form-fitting one-size-fits-all fit.

Challenges from vacuum systems is that patients often don't like the feel and simply don't feel as secure using their prosthetic devices as compared to more traditional prosthetic liner systems. Vacuum systems provide a stronger fit than suction systems, but with vacuum systems, if the vacuum is too tight and restrictive, tissue damage can occur in the residual limb. Vacuum and pump systems are also used for pads placed in the hard socket, for example, air bladders that can be pumped out or released to provide for fit within the hard socket. The problem with all vacuum and pump systems is that a tear, even a microtear, causes immediate failure. Changes in barometric pressure also wreak havoc on pump systems and air bladders.

The other problem with pump systems, such as air bladders, is that the amputee has a harder time getting a feel for the environment around him or her. Just as how most people can feel the texture of the ground under their feet even when wearing shoes (being able to tell whether they are walking on gravel, or grass, or a concrete surface, for example), the amputee can discern a lot of information through their prosthetic device from the environment when he or she has a good fit. When the fit is poor, this sensory information is lost. Likewise, this sensory information is greatly reduced through any air system, which does not-transmit this type of sensory information through the air gap in the pump systems as well as the prosthetic device, the hard socket, and the liner, which are all made of more solid or semi-solid materials than an air bladder.

The modeling of a perfect fit prosthetic socket is complex and unique for each patient. The modeling for sockets, and test case uses, is currently being performed in Prof. Hugh Herr's Biomechatronics Laboratory at MIT, by Prof Herr, the founder of iWalk, now Biom, recently acquired in part by Otto Bock, with a custom fit socket (U.S. Ser. No. 13/836,835); however, this is a relatively static system with respect to fit. Humans are dynamic, particularly when in motion, and undergo marked changes from an initial prosthetic fitting, even with state-of-the-art modeling and design.

Smart materials have found uses as sensors, such as using dielectric elastomeric actuators (DEAs) as sensors and self-sensors. Dielectric materials are poor conductors of electricity but are good at supporting electrostatic fields, so they act as capacitors. SRI international, Artificial Muscle Inc., and Stretch Sense/University of Auckland have found that DEAs have the potential for sensing and for self-sensing, where self-sensing is sensing an electrical property of the actuator itself (U.S. Pat. Nos. 8,860,336, 7,521,840, 7,595,580, 6,768,246). The state of a DEA can be determined by sensing the capacitance between the electrodes. Due to the high voltages (kV range) applied to the electrodes which are necessary to actuate a DEA, implementing capacitive self-sensing is not as simple as applying the capacitive sensing techniques commonly applied in other fields. The methodology for self-sensing in DEAs is very complex.

Currently, to manage and provide fit for the amputee, the diagnostic process during fitting is feedback from what the patient is saying to the prosthetist. Currently, there is no actual physical feedback data. For areas that are innervated, or if the patient's nerves are comprised, which often occurs in diabetes patients, the fit is difficult to get just right in the prosthetist's office. Poor fit leads to discomfort and tissue breakdown.

### SUMMARY

In addition to the shape-morphing abilities of electroactive polymers (also referred to as EAPs) developed by previous inventions [U.S. patent application Ser. No. 14/476,646, U.S. patent application Ser. No. 13/843,959, U.S. Pat. Nos. 8,088,453, 7,935,743, and 5,736,590], and these shape-morphing EAPs' sensing capabilities [PCT/US2015/058951], the EAPs in the instant invention are also made to be capable of attenuating force and with excellent viscoelastic properties. Particularly when comprising a multi-modal (or multi-layer) EAP, the force attenuation can be further enhanced. The EAPs and EAP-based systems in the instant invention will shape-morph, sense pressure, and attenuate force to provide for a perfect, extremely comfortable fit throughout the day, and will also provide for continuously good sensory feedback to the amputee, through both the maintenance of perfect fit and through these EAPs being semi-solid materials rather than gaseous. The EAPs and EAP-based systems in the instant invention are variable resistors which sense mechanical pressure, and are much more simplified systems than DEA capacitor-based systems.

Traditional liner materials have problems with creep and hysteresis. The EAPs and EAP-based systems in the instant invention, in addition to being able to shape-morph, sense pressure, and attenuate force, also have good creep resistance, good elasticity, and low hysteresis effects. These attributes make these EAPs and EAP-based systems in the instant invention ideally suited for prosthetic liners and for pads in the hard socket, particularly during ambulation (walking and running).

Described herein is electroactive polymers or electroactive ionic polymers (used interchangeably herein and both can be referred to as EAPs) that controllably contract and expand at low voltage to provide for a shape-morphing system, that also can sense pressure (from gentle touch to high impact), and that can also attenuate force. In particular, when a multi-modal/multi-layer EAP system is used, the force attenuation through the system can be attenuated through greater lateral dispersion of force. The EAP and EAP-based systems in the instant invention also have good creep resistance, good elasticity, and low hysteresis effects.

In some embodiments, protective gear and sports padding, such as for helmets, including the EAP-based system, are described. In some embodiments, protective gear and sports apparel, such as for shoe insoles, including the EAP-based system are described. In some embodiments, prosthetic liners, including the EAP-based system are described. In some embodiments, pads in prosthetic hard sockets, including the EAP-based system are described. In some embodiments, diagnostics for prosthetic fit, including the EAP-based system are described. In some embodiments, compression equipment, such as compression boots for diabetic patients, military anti-shock trousers (MAST, also called pneumatic anti-shock garments or PASG) for trauma patients, compression bandages or tapes, and compressive therapies, including the EAP-based system are described.

In the instant invention, an EAP-based system, in a thin layer, could be used to diagnose fit in the care of a prosthetist, using the sensing abilities of the thin EAP layer, which could be pixelated, to provide for pressure mapping, and thus providing real-time data for the fitting of the patient to the prosthetic device. Real-time feedback on fit with the prosthetist will lead to improved fit and much better limb health. This type of pixelated EAP-based thin or thick film could also be used for feedback in compression equipment, such as compression boots for diabetic patients, military anti-shock trousers (MAST, also called pneumatic anti-shock garments or PASG) for trauma patients, compression bandages or tapes, and other compressive therapies and applications. This type of pixelated EAP-based thin or thick film could also be used as “skin” for prosthetic and robotic hands and arms, and for fingertip areas in prosthetic and robotic hands (or grippers, also known as end effectors in robotics).

In one aspect, protective gear or sports padding comprising one or more EAP-based systems disclosed herein is described, where the EAP-based padding uses the shape-morphing features of the EAP-based system to fit each individual's specific anthropometry. The sensing ability of the EAP-based system senses the number and severity of impacts. The sensing feature can be compiled and communicated for feedback by a compilation log feature in the helmet, or the impact data (number of impacts and severity) could be transmitted via a Bluetooth adapter, for example, to a computer or other electronic device, or a combination thereof. This way the player, the parents, the team's doctor, and/or the coach have a diagnostic tool to help in the assessment of the player's potential concussive condition, where not only one high impact is of concern, but several lower impacts within a short time span are also of concern and can be just as detrimental. The force attenuation feature of the EAP-based system attenuates the force from impacts to prevent and reduce injury and death.

In one aspect, protective gear or sports apparel comprising one or more EAP-based systems disclosed herein is described. The shape-morphing features of the EAP-based system provides perfect fit in the shoe insole. For example, shape-morphing within the EAP-based system of the shoe insole provides perfect arch support for each individual. The sensing features of the EAP-based system of the shoe insole provides for feedback of the number of steps and the mechanical pressure profile of the foot strike, which can be pixelated, or a combination thereof. The force attenuation of the EAP-based system provides for an extremely comfortable feeling in the shoe insole and potential better health for the feet. Especially for diabetic patients, the sensing feature of the EAP-based system in the shoe insole provides feedback for areas that are becoming prone to ulceration (dead zone areas) before tissue damage occurs, while the non-Newtonian force attenuation of the EAP-based system provides for a massage-like effect from the shoe insole, potentially improving the comfort and health of the diabetic foot. The sensing feature can be compiled and communicated for feedback by a compilation log feature in the shoe, or the data (number of steps and pressure mapping of the foot) could be transmitted via a Bluetooth adapter, for example, to a computer or other electronic device, or a combination thereof.

In one aspect, prosthetic liners comprising one or more shape-morphing systems disclosed herein are described, where the EAP-based system is used for the prosthetic liner. The shape-morphing features of the EAP-based system provides perfect fit between the residual limb and the hard socket of the prosthetic device. The sensing features of the EAP-based system of the prosthetic liner provides for feedback of the fit, allowing for the liner to be continually and automatically adjusted for automatic perfect fit throughout the day. The force attenuation and the viscoelasticity of the EAP-based system provide for an extremely comfortable feeling in the prosthetic liner, with excellent rebound during ambulation. This provides for an extremely comfortable perfect fit and potentially for much better health of the residual limb.

In one aspect, pads in the hard socket of the prosthetic device comprising one or more EAP-based systems disclosed herein are described, where the EAP-based system is used for the pads in the hard socket. The shape-morphing features of the EAP-based system provide perfect fit for the residual limb within the hard socket of the prosthetic device. The sensing features of the EAP-based system of the prosthetic liner provide for feedback of the fit, allowing for the shape-morphing pads to be continually and automatically adjusted for automatic perfect fit throughout the day. The force attenuation and the viscoelasticity of the EAP-based system provide for an extremely comfortable feeling within the hard socket, with excellent rebound during ambulation. This provides for an extremely comfortable perfect fit and potentially for much better health of the residual limb. These shape-morphing padded systems can be provided as a kit for the prosthetist to place within the hard socket during fitting of the patient. Alternatively, these shape-morphing padded systems can be embedded or incorporated as part of the hard socket during the construction or production of the hard socket itself.

In one aspect, diagnostics for prosthetic fit comprising one or more EAP-based systems disclosed herein is described, where the EAP-based system is used for the diagnostic device for prosthetic fit. The sensing features of the EAP-based system of the diagnostic device, comprising a thin layer or thin sheath of the shape-morphing materials, provide for feedback of the fit for the patient and his or her prosthetist, allowing the prosthepist a data-driven diagnostic device to help with fitting the patient for their prosthetic liner and prosthetic device. In the instant invention, an EAP-based system, in a thin layer, could be used to diagnose fit in the care of a prosthetist, using the sensing abilities of the thin EAP layer, which could be pixelated, to provide for pressure mapping, and thus providing real-time data for the fitting of the patient to the prosthetic device. The force attenuation and the viscoelasticity of the EAP-based system provides for a more durable diagnostic device. The sensing feature can be compiled and communicated for feedback by a compilation log feature in the device, or the data (pressure mapping of the residual limb being fitted in the prosthetic device) could be transmitted via a Bluetooth adapter, for example, to a computer or other electronic device, or a combination thereof.

In one aspect, compressive equipment comprising one or more EAP-based systems disclosed herein is described, where the EAP-based system is used for the compressive equipment or therapy. The shape-morphing features of the EAP-based system provide a good compressive fit for the patient using the compressive equipment, where the shape-morphing could be dynamic, such as wave-like compression to massage and remove fluid from the limb of a diabetes patient or a lymphatic-compromised patient who is suffering from swelling of the extremities. The sensing features of the EAP-based system of the prosthetic liner provide for feedback of the fit. The force attenuation of the EAP-based system provides for an additional massage-like effect from the compressive equipment, improving the comfort and saving limbs in the case of the diabetic compressive boot, and lives in the case of MAST compressive equipment. This three-fold EAP system (shape-morphing, sensing, and force attenuation) would also be beneficial for compressive tapes and other compressive equipment, beyond medical applications.

In one aspect, an electroactive polymer-based system is described, including:

a first electrode;

a second electrode counter to the first electrode and spaced apart from the first electrode;

an ionically conductive fluid; and

an actuator electronically connected to the first electrode and in fluidic communication with the second electrode, and including:
a first electroactive ionic polymer layer including a first electroactive ionic polymer; and
a second electroactive ionic polymer layer including a second electroactive ionic polymer; wherein the first and second electroactive polymers are selected to expand or contract on application of an electrical potential; wherein the Shore O durometer value of the second electroactive ionic polymer is higher than that of the first electroactive ionic polymer; and wherein the first and second electroactive ionic polymer layers are configured to transfer the force applied onto the first electroactive ionic polymer layer to the second electroactive ionic polymer layer to be attenuated.

In any one of the embodiments described herein, the first and second electroactive ionic polymer layers are in direct contact with each other or in close proximity to each other.

In any one of the embodiments described herein, the first and second electroactive ionic polymer layers are separated by a soft or elastic layer.

In any one of the embodiments described herein, the difference of Shore O durometer values between the first and second electroactive ionic polymer layers is about 2-60.

In any one of the embodiments described herein, the first electroactive ionic polymer has a cross-link density of at least about 1.5%-6% vol/wt of cross-linking agent/linear monomers.

In any one of the embodiments described herein, the second electroactive ionic polymer has a cross-link density of less than about 1.5% vol/wt of cross-linking agent/linear monomers.

In any one of the embodiments described herein, the second electroactive ionic polymer has a cross-link density of about 0.50%, 0.75%, 1.00%, 1.25%, 1.50%, 1.60%, 1.75%, 1.80%, 2.00%, 2.25%, or 2.50% higher for each cross-linking agent than that of the first electroactive ionic polymer, the second electroactive ionic polymer has a cross-link density in a range bounded by any two values disclosed herein.

In any one of the embodiments described herein, the second electroactive ionic polymer layer has a shape selected from the group consisting of conical, half-ovoid, ovoid, sheet, pad, sphere, cylinder, cone, pyramid, prism, spheroid ellipse, ellipsoid, rectangular prism, toroid, parallelepiped, rhombic prism shapes and a combination thereof.

In any one of the embodiments described herein, the second electroactive ionic polymer layer has a shape selected from the group consisting of a conical shape, a half-ovoid shape, an ovoid shape, and a combination thereof.

In any one of the embodiments described herein, the first electroactive ionic polymer layer has a shape reciprocal to the shape of the second electroactive ionic polymer layer.

In any one of the embodiments described herein, the electroactive polymer-based system further includes one or more electrically conducting layers in electrical contact with the actuator and the first electrode.

In any one of the embodiments described herein, the electroactive polymer-based system comprises a first and second electrically conducting layers in electrical contact with the first and second electroactive ionic polymer layers, respectively.

In any one of the embodiments described herein, the electrically conducting layer comprises an array of a plurality of electrically conducting areas.

In any one of the embodiments described herein, the electroactive polymer-based system further includes a fluid reservoir in fluidic communication with the first and second electroactive ionic polymers and connected to the second electrode. In any one of the embodiments described herein, the fluid reservoir is in the second electroactive ionic polymer layer.

In any one of the embodiments described herein, the first and/or second electroactive ionic polymers are each independently selected from the group consisting of polymethacrylic acid, poly-2-hydroxyethyl methacrylate, poly(vinyl alcohol), ionized poly(acrylamide), poly(acrylic acid), poly(acrylic acid)-co-(poly(acrylamide), poly(2-acrylamide-2-methyl-1-propane sulfonic acid), poly(methacrylic acid), poly(styrene sulfonic acid), quarternized poly(4-vinyl pyridinium chloride), poly(vinylbenzyltrimethyl ammonium chloride), sulfonated poly(styrene-b-ethylene-co-butylene-b-styrene), sulfonated poly(styrene), and a combination thereof.

In any one of the embodiments described herein, the first and/or second electroactive ionic polymers are cross-linked with one or more cross-linking polymer agents each selected from the group consisting of a poly(dimethylsiloxane) (PDMS) dimethacrylate chain, a poly(ethylene glycol) dimethacrylate chain, an ethylene glycol dimethacrylate, 1,1,1-trimethylolpropane trimethacrylate, and a combination thereof.

In any one of the embodiments described herein, the first electroactive ionic polymer is cross-linked with one or more first cross-linking polymer agents which is elastomeric or provides elasticity.

In any one of the embodiments described herein, the first cross-linking polymeric agent comprising a poly(dimethylsiloxane) (PDMS) dimethacrylate chain.

In any one of the embodiments described herein, the second electroactive ionic polymer is cross-linked with a second cross-linking polymeric agent selected from the group consisting of a poly(ethylene glycol) dimethacrylate chain, an ethylene glycol dimethacrylate, 1,1,1-trimethylolpropane trimethacrylate, and a combination thereof; or wherein the second electroactive ionic polymer is cross-linked with the second cross-linking polymeric agent and a poly(dimethylsiloxane) (PDMS) dimethacrylate chain; or wherein the second electroactive ionic polymer is cross-linked with a higher level of a cross-linking polymeric agent than the cross-linking polymeric agent is selected from the group consisting of a poly(dimethylsiloxane) dimethacrylate, poly(ethylene glycol) dimethacrylate, an ethylene glycol dimethacrylate, 1,1,1-trimethylolpropane trimethacrylate, and a combination thereof.

In any one of the embodiments described herein, the second electroactive ionic polymer is cross-linked with a second cross-linking polymeric agent selected from the group consisting of a poly(dimethylsiloxane) dimethacrylate, a poly(ethylene glycol) dimethacrylate chain, an ethylene glycol dimethacrylate, 1,1,1-trimethylolpropane trimethacrylate, and a combination thereof; where the second electroactive ionic polymer is cross-linked at a higher level than that of the first electroactive ionic polymer

In another aspect, an electroactive polymer-based system is described, including:

one or more first electrodes;

a second electrode counter to the first electrode and spaced apart from the first electrode;

an ionically conductive fluid; and

an actuator electronically connected to the first electrodes and in fluidic communication with the second electrode, and including
an electroactive ionic polymer layer comprising an electroactive ionic polymer selected to expand or contract on application of an electrical potential; and
an array of a plurality of isolated conductive areas each in electric communication with a plurality of areas of the electroactive ionic polymer layer; wherein the plurality of isolated conductive areas comprises at least one or more first isolated conductive areas in electric communication with the one or more first electrodes independent from other isolated conductive areas such that the areas of the electroactive ionic polymer layer in electric communication with the first isolated conductive areas are capable of being actuated independently.

In any one of the embodiments described herein, the electroactive polymer-based system further includes one or more third electrodes; the plurality of isolated conductive areas comprises at least one or more second isolated conductive areas in electric communication with the one or more third electrodes independent from other isolated conductive areas such that the areas of the electroactive ionic polymer layer in electric communication with the second isolated conductive areas are capable of being actuated independently.

In yet another aspect, a liner for securing a limb in a prosthetic device or a prosthetic socket is described, including:

a flexible layer configured to surround a limb of a patient or conform to the inside circumference of a prosthesis; and

at least one electroactive polymer-based system of any one of the embodiments described herein embedded in the flexible layer and configured to secure or engage a limb of a patient.

In any one of the embodiments described herein, the flexible layer is made of silicone.

In any one of the embodiments described herein, the liner or prosthetic socket comprises a plurality of the electroactive polymer-based system of any one of the embodiments described herein and embedded in the flexible layer; wherein the electroactive polymer-based systems are fluidically isolated from each other and arranged around the limb of a patient to secure the limb.

In any one of the embodiments described herein, the prosthesis has a hard body and upon the application of an electrical potential to the first electrode, the actuator is configured to expand against the hard body towards the limb of the patient.

In yet another aspect, a shoe insole including the electroactive polymer-based system of any one of the embodiments described herein is described.

In yet another aspect, a protective gear including the electroactive polymer-based system of any one of the embodiments described herein is described.

In any one of the embodiments described herein, the protective gear is a helmet.

In yet another aspect, a compression equipment including the electroactive polymer-based system of any one of the embodiments described herein is described.

In any one of the embodiments described herein, the compression equipment is a compression boot for diabetic patients, a military anti-shock trouser (MAST, also called pneumatic anti-shock garments (PASG)) for trauma patients, a compression bandage, a compression tape, or a compressive therapy

In any one of the embodiments described herein, the EAP-based system includes two or more layers of EAPs which exhibit force attenuation properties. Even without the layering effect, these EAPs and EAP-based systems are force attenuating, however, due to the semi-solid non-Newtonian behavior. These EAPs and EAP-based systems can be enhanced with carbon fibers, filaments, weaves, and nanofibers/filaments, metal particles, metal wires, metal meshes, metal nano-particles, wires, or meshes, or a combination thereof, to provide for better dispersal of electric charge throughout the EAP and application-specific viscoelastic behavior and anisotropy. These EAPs and EAP-based systems can be customized to meet the toughness, flexibility, and strength required for specific applications in environments as varied as space, deep oceans, prosthetics (repetitive loading and long duration use), and helmets (high-impact sports and military use).

The EAPs and EAP-based systems in the instant invention integrate three key head health components into one system by combining impact attenuation with collision severity sensing and shape morphing to provide for injury protection, real time head impact sensing, and individualized comfort that can be tailored for use whether on the playing field or on the battlefield. First, the EAPs and EAP-based systems in the instant invention, even in an uncharged state, resist impact due to the non-Newtonian semi-solid behavior and the multi-modality of these novel smart materials. Second, these EAPs and EAP-based systems are variable resisters and change their impedance when exposed to force. This unique electro-mechanical phenomenon allows these EAPs and EAP-based systems to sense the occurrence, direction, and severity of an impact to the head, and when coupled with in-helmet electronics that have been demonstrated as practical in the elite athlete setting, the material can be used as a real time on-field concussion screening tool. When used in conjunction with sideline and locker-room concussion diagnostics, these EAPs and EAP-based systems have the potential to assist in the concussion diagnosis process. Third, these EAPs and EAP-based systems can actively shape-morph, thus advancing impact attenuation over currently available materials by being able to be tuned on the sideline for optimal stiffness, shape, and comfort without being removed from the athlete's head. The EAP in the instant invention contracts or expands with electric input, and returns to its original size (uncharged state) when the electric input is turned off. The practical application is that a player presses a helmet-mounted button to apply a very low power current, which contracts the helmet liners before donning the helmet, and once donned releases the button to expand the liners to fit snugly to the player's unique anthropometry. This feature can not only benefit the elite athlete with perfect fit on well financed teams, but also youth and amateur athletes on teams with modest equipment budgets who rely on a one-size-fits-all approach. As used herein, shape-morphing, sensing, and force attenuating system, EAP-based system, and electroactive polymer-based system are used interchangeably.

It is contemplated that any embodiment disclosed herein may be properly combined with any other embodiment disclosed herein. The combination of any two or more embodiments disclosed herein is expressly contemplated.

As used herein, the use of the phrase “polymer” includes, but is not limited to, the homopolymer, copolymer, terpolymer, random copolymer, and block copolymer. Block copolymers include, but are not limited to, block, graft, dendrimer, and star polymers. As used herein, copolymer refers to a polymer derived from two monomeric species; similarly, a terpolymer refers to a polymer derived from three monomeric species. The polymer also includes various morphologies, including, but not limited to, linear polymer, branched polymer, random polymer, crosslinked polymer, and dendrimer systems. As an example, polyacrylamide polymer refers to any polymer including polyacrylamide, e.g., a homopolymer, copolymer, terpolymer, random copolymer, block copolymer or terpolymer of polyacrylamide. Polyacrylamide can be a linear polymer, branched polymer, random polymer, crosslinked polymer, or a dendrimer of polyacrylamide.

As used herein, the phrase “ionic polymer” refers to any polymer which has one or more ionizable groups. Non-limiting examples of the ionic polymers include a polymer of one or more ionic-group containing monomers. Other non-limiting examples of the ionic polymers include a polymer which has one or more ionic groups at any position of the polymeric chain. As used herein, the phrase “electroactive ionic polymer” refers to any polymer which has one or more ionizable groups and can shape-change, e.g., expand or contract, upon the application of voltage.

Unless otherwise defined, used or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein.

Although the terms “first”, “second”, “third”, etc., may be used herein to describe various elements, these elements are not to be limited by these terms. These terms are simply used to distinguish one element from another. Thus, a first element, discussed below, could be termed a second element without departing from the teachings of the exemplary embodiments. Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. Further still, in this disclosure, when an element is referred to as being “linked to,” “on,” “connected to,” “coupled to,” “in contact with,” etc., another element, it may be directly linked to, on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise. Additionally, the terms, “includes,” “including,” “comprises” and “comprising,” specify the presence of the stated elements or steps but do not preclude the presence or addition of one or more other elements or steps.

### DESCRIPTION OF THE DRAWINGS

The invention is described with reference to the following figures, which are presented for the purpose of illustration only and are not intended to be limiting. In the Drawings:

FIG. 1A is a schematic view of an encapsulated electroactive polymer (EAP)-based system, according to one or more embodiments described herein.

FIG. 1B is a schematic view of an electroactive polymer (EAP)-based shape-morphing system, sensing, and force attenuating system, which is multi-modal or multi-layer, where the lower EAP layer is of a higher cross-link density and thus higher durometer than the upper EAP layer, and the lower EAP layer has a conical shape.

FIG. 2 is a schematic view of an EAP-based shape-morphing system, sensing, and force attenuating system, which is multi-modal or multi-layer, where the lower EAP layer is of a higher cross-link density and thus higher durometer than the upper EAP layer, and the lower EAP layer has a half-ovoid shape.

FIG. 3 is a schematic view of an encapsulated EAP-based shape-morphing, sensing, and force attenuating system as a pad for the prosthetic liner or socket, according to one or more embodiments described herein.

FIG. 4 is a schematic view of an encapsulated EAP-based shape-morphing, sensing, and force attenuating system for shoe insoles, for example, with conductive layers above and below the EAP layer, where the conductive layer(s) can be pixelated.

FIG. 5 is a cross-sectional view of an encapsulated EAP-based shape-morphing, sensing, and force attenuating system within a flexible prosthetic liner, according to one or more embodiments described herein.

FIG. 6 is a schematic view of an encapsulated EAP-based shape-morphing, sensing, and force attenuating system within a flexible prosthetic liner, according to one or more embodiments described herein.

FIG. 7 is a schematic view of an encapsulated EAP-based shape-morphing, sensing, and force attenuating system within a prosthetic hard socket, according to one or more embodiments described herein.

FIG. 8 is a schematic view of an encapsulated EAP-based shape-morphing, sensing, and force attenuating system as a band in the prosthetic liner or prosthetic socket, according to one or more embodiments described herein.

FIG. 9 is a schematic view of an encapsulated EAP-based shape-morphing, sensing, and force attenuating system as a band in the prosthetic socket, according to one or more embodiments described herein.

FIG. 10 is a schematic view of an encapsulated EAP-based EAP shape-morphing, sensing, and force attenuating system as a band in the prosthetic liner, according to one or more embodiments described herein.

FIG. 11 is a schematic view of an encapsulated EAP-based EAP shape-morphing, sensing, and force attenuating system as a compression boot, according to one or more embodiments described herein.

FIG. 12 is a transparent view of an encapsulated EAP-based shape-morphing, sensing, and force attenuating system as a compression boot, according to one or more embodiments described herein.

FIG. 13 is a schematic view of an encapsulated EAP-based shape-morphing, sensing, and force attenuating system as compression tape, according to one or more embodiments described herein.

FIG. 14 illustrates drop tower data comparing EAPs in the instant invention (EAP samples in the instant invention are the RasFlex series) to traditional padding in Xenith and Riddell football helmets, according to one or more embodiments described herein.

FIG. 15 illustrates the compression testing of EAP Sample LA_12 using an Instron® Model 4466 Universal Testing Machine, at a speed of 3 mm/min with a peak compressive force of 174.964 N, according to one or more embodiments described herein.

FIG. 16 illustrates cyclic stress-strain testing of EAP Sample LR_97_BJ using a Universal Testing Machine to 50% elongation, according to one or more embodiments described herein.

### DETAILED DESCRIPTION

In one aspect, an electroactive polymer-based system is described, including:

a first electrode;

a second electrode counter to the first electrode and spaced apart from the first electrode;

an ionically conductive fluid; and

an actuator electronically connected to the first electrode and in fluidic communication with the second electrode, and comprising
a first electroactive ionic polymer layer comprising a first electroactive ionic polymer; and
a second electroactive ionic polymer layer comprising a second electroactive ionic polymer; wherein the first and second electroactive polymers are selected to expand or contract on application of an electrical potential; wherein the durometer value of the second electroactive ionic polymer is higher than that of the first electroactive ionic polymer; and wherein the first and second electroactive ionic polymer layers are configured to transfer the force applied onto the first electroactive ionic polymer layer to the second electroactive ionic polymer layer to be attenuated.

The phrase “durometer value” or “durometer,” as used herein, refers to the measurement of hardness of a material, where the numerical value, between 0 and 100, defines the hardness or softness of a material. Higher numbers indicate harder materials; lower numbers indicate softer materials. Durometer is typically used as a measure of hardness in polymers, elastomers, and rubbers. Durometer measures the depth of an indentation in the material created by a given force on a standardized pressure foot. A standard way of determining the durometer value of an EAP layer is to place an EAP sample that is at least 6.4 mm thick on top of a hard surface, and then used the durometer instrument to measure the durometer value by pressing the pressure foot on the top area of the EAP and noting the durometer value on the scale. Durometer comes in 12 different scales. The EAPs in the instant invention are typically measured using the Shore O and the Shore OO scale, to determine hardness in these relatively soft materials.

In certain embodiments, the actuator is apart from the second electrode. In certain embodiments, the electroactive polymer-based system further includes an electrically conducting backing or a conductive layer disposed along and in electrical contact with a surface of the actuator. The conducting backing or a conductive layer may be bonded to a surface of the actuator. Any level/manner of bonding is contemplated.

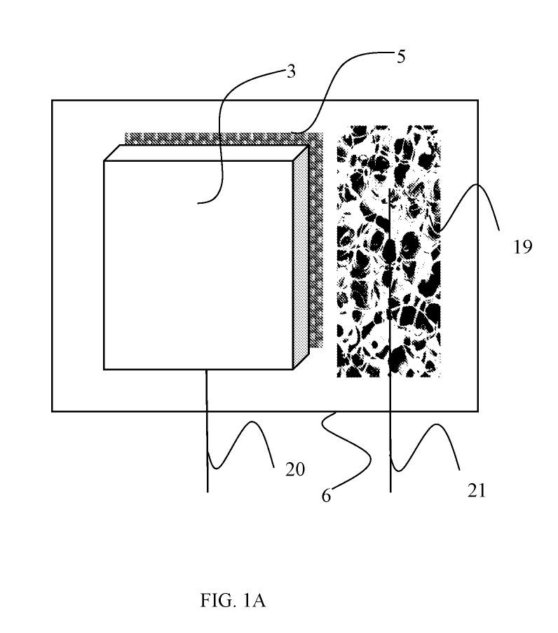

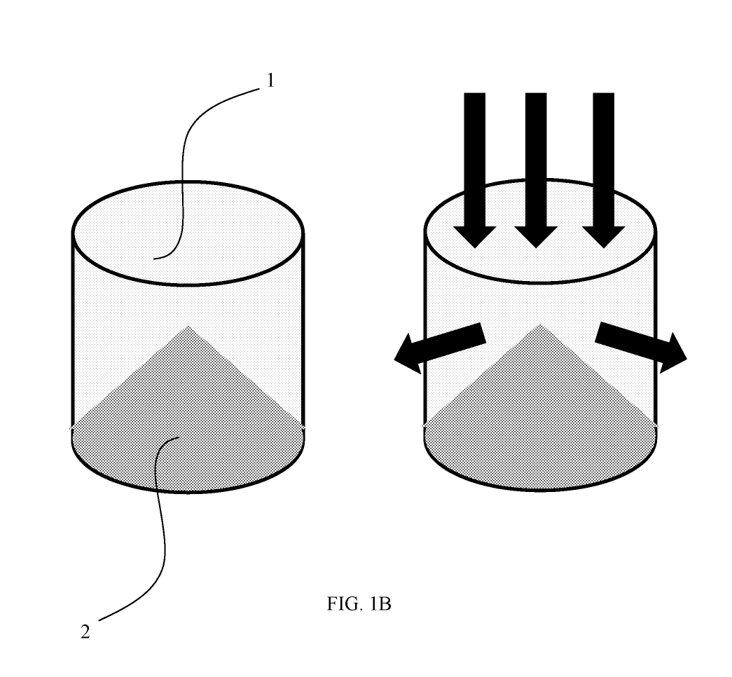

The EAP-based system disclosed herein is now described with reference to FIG. 1A. FIG. 1A shows an encapsulated electroactive polymer (EAP)-based system encapsulated in a flexible encapsulating coating 6. The EAP-based system includes a first electrode 20, a second electrode 21 counter to the first electrode and spaced apart from the first electrode 20, an ionically conductive fluid in the fluidic reservoir 19, and an actuator 3 including one or more EAP shape-morphing, sensing, and force attenuating layer which is electronically connected to the first electrode 20 through a conductive layer 5 and in fluidic communication with the second electrode 21. The EAP polymers are selected to expand or contract on application of an electrical potential. The fluidic reservoir 19 can be located in any other part of the EAP-based system. In some embodiments, the fluidic reservoir 19 can be between the upper and lower EAP layers as described below. In some embodiments, the fluidic reservoir 19 can be the second EAP layer or part of the second EAP layer.

In some embodiments, because these EAPs, in order to be electroactive, need to be moist and contain an electrolyte, the electroactive material may be further swollen with an electrolyte solution or electrolyte gel formulation. Other suitable materials and compositions for the electroactive material are described in U.S. Pat. Nos. 8,088,453, 7,935,743, and 5,736,590 and U.S. Ser. Nos. 13/843,959 and 14/476,646, the contents of which are expressly incorporated by reference.

In some embodiments, the actuator 3 described in FIG. 1A includes a first electroactive ionic polymer layer comprising a first electroactive ionic polymer; and a second electroactive ionic polymer layer comprising a second electroactive ionic polymer; wherein the first and second electroactive polymers are selected to expand or contract on application of an electrical potential; wherein the durometer value of the second electroactive ionic polymer is higher than that of the first electroactive ionic polymer; and wherein the first and second electroactive ionic polymer layers are configured to transfer the force applied onto the first electroactive ionic polymer layer to the second electroactive ionic polymer layer to be attenuated.

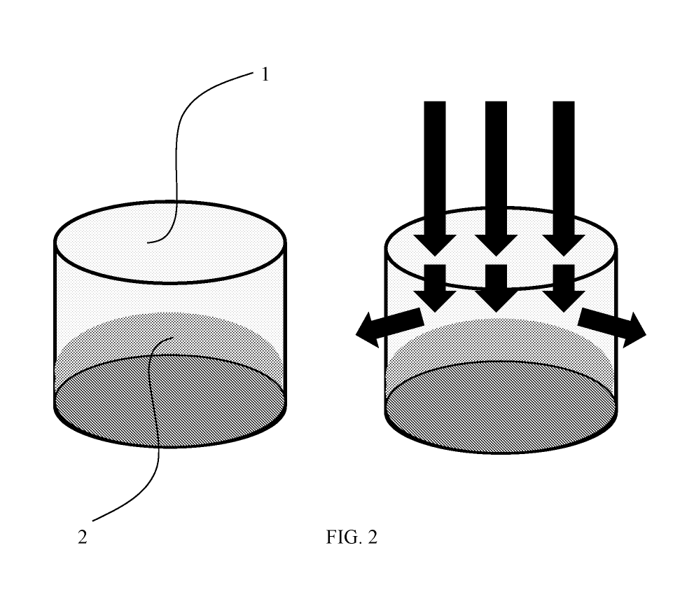

The multi-layer EAP-based actuator disclosed herein is now described with reference to FIGS. 1B and 2. FIG. 1B shows an electroactive polymer (EAP)-based system (left hand side), where the multi-modality for force attenuation is displayed (right hand side). The EAP-based system includes two EAP layers (1 and 2). The lower EAP layer 2 is of a higher cross-link density and thus higher durometer than the upper EAP layer 1. This multi-modal/multi-layer EAP system can have a conductive layer (which may be electrically connected to an electrode) above and below the EAP system for sensing and shape-morphing, and can be encapsulated, with a flexible silicone layer, for example. The lower layer 2 (e.g., a more cross-linked layer) would be closer to the head for helmets, bottom of the feet for shoe soles, or to any part of the body for padding. The upper layer (less cross-linked layer) would be closer to the outside environment. The lower EAP layer has a conical shape and the upper EAP layer has a reciprocal shape. This design allows for more force to be deflected laterally rather than through the system. However, any other shapes known in the art are contemplated.

FIG. 2 shows an electroactive polymer (EAP)-based system (left hand side), where the multi-modality for force attenuation is displayed (right hand side). The lower EAP layer 2 is of a higher cross-link density and thus higher durometer than the upper EAP layer 1. This multi-modal EAP system can have a conductive layer (which may be electrically connected to an electrode) above and below the EAP system for sensing and shape-morphing, and can be encapsulated, with a flexible silicone layer, for example. The lower layer 2 (more cross-linked layer) would be closer to the head for helmets, bottom of the feet for shoe soles, or to any part of the body for padding. The upper layer 1 (less cross-linked layer) would be closer to the outside environment. The lower EAP layer 2 has a half-ovoid shape and the upper EAP layer has a reciprocal shape. This design allows for more force to be deflected laterally rather than through the system. However, any other shapes known in the art are contemplated.

Non-limiting examples of the shapes for the lower EAP layer (also referred to as the second electroactive ionic polymer layer) include a shape selected from the group consisting of conical, half-ovoid, ovoid, sheet, pad, sphere, cylinder, cone, pyramid, prism, spheroid ellipse, ellipsoid, rectangular prism, toroid, parallelepiped, rhombic prism and a combination thereof. In some embodiments, the upper EAP layer (also referred to as the first electroactive ionic polymer layer) has a shape reciprocal to the shape of the lower EAP layer. In some embodiments, the two EAP layers (1 and 2) are in direct contact with each other or in close proximity to each other so that the force applied onto the upper layer 1 (lower durometer value) is transferred to the lower layer (higher durometer value) to be attenuated, and the force is dispersed laterally. In other embodiments, the two EAP layers (1 and 2) are not in direct contact but are separated by a soft or elastic layer so that through the soft or elastic layer, the force applied onto the upper layer 1 (lower durometer value) is transferred to the lower layer (higher durometer value) to be attenuated. The soft or elastic layer can be porous to allow electrolyte to pass through to maintain fluid communication. In some embodiments, the soft or elastic layer can serve as the fluidic reservoir 19 described in FIG. 1A.

In some embodiments, the lower EAP layer (also referred to as the second EAP layer) has a durometer value of about at least 2, 3, 4, 5, 6, 7, 8, 9, 10, 15, 20, 25, 30, 35, 40, 45, 50, 55, 60 higher than that of the upper EAP layer (also referred to as the first EAP layer). In some embodiments, the durometer value difference between the first and second EPA layers is in a range bounded by any two numbers disclosed above.

In some embodiments, the durometer value difference between the first and second EPA layers is about 2-60, 2-55, 2-50, 2-45, 2-40, 2-35, 2-30, 2-25, 2-20, 2-15, 2-10, 2-9, 2-8, 2-7, 2-6, 2-5, 2-4, 2-3. In some embodiments, the durometer value difference between the first and second EPA layers is about 3-60, 3-55, 3-50, 3-45, 3-40, 3-35, 3-30, 3-25, 3-20, 3-15, 3-10, 3-9, 3-8, 3-7, 3-6, 3-5, or 3-4. In some embodiments, the durometer value difference between the first and second EPA layers is about 4-60, 4-55, 4-50, 4-45, 4-40, 4-35, 4-30, 4-25, 4-20, 4-15, 4-10, 4-9, 4-8, 4-7, 4-6, or 4-5. In some embodiments, the durometer value difference between the first and second EPA layers is about 5-60, 5-55, 5-50, 5-45, 5-40, 5-35, 5-30, 5-25, 5-20, 5-15, 5-10, 5-9, 5-8, 5-7, or 5-6. In some embodiments, the durometer value difference between the first and second EPA layers is about 6-60, 6-55, 6-50, 6-45, 6-40, 6-35, 6-30, 6-25, 6-20, 6-15, 6-10, 6-9, 6-8, or 6-7. In some embodiments, the durometer value difference between the first and second EPA layers is about 7-60, 7-55, 7-50, 7-45, 7-40, 7-35, 7-30, 7-25, 7-20, 7-15, 7-10, 7-9, or 7-8. In some embodiments, the durometer value difference between the first and second EPA layers is about 8-60, 8-55, 8-50, 8-45, 8-40, 8-35, 8-30, 8-25, 8-20, 8-15, 8-10, or 8-9. In some embodiments, the durometer value difference between the first and second EPA layers is about 9-60, 9-55, 9-50, 9-45, 9-40, 9-35, 9-30, 9-25, 9-20, 9-15, or 9-10. In some embodiments, the durometer value difference between the first and second EPA layers is about 10-60, 10-55, 10-50, 10-45, 10-40, 10-35, 10-30, 10-25, 10-20, or 10-15. In some embodiments, the durometer value difference between the first and second EPA layers is about 15-60, 15-55, 15-50, 15-45, 15-40, 15-35, 15-30, 15-25, or 15-20. In some embodiments, the durometer value difference between the first and second EPA layers is about 20-60, 20-55, 20-50, 20-45, 20-40, 20-35, 20-30, or 20-25. In some embodiments, the durometer value difference between the first and second EPA layers is about 25-60, 25-55, 25-50, 25-45, 25-40, 25-35, or 25-30. In some embodiments, the durometer value difference between the first and second EPA layers is about 30-60, 30-55, 30-50, 30-45, 30-40, or 30-35. In some embodiments, the durometer value difference between the first and second EPA layers is about 35-60, 35-55, 35-50, 35-45, or 35-40. In some embodiments, the durometer value difference between the first and second EPA layers is about 40-60, 40-55, 40-50, or 40-45. In some embodiments, the durometer value difference between the first and second EPA layers is about 45-60, 45-55, or 45-50. In some embodiments, the durometer value difference between the first and second EPA layers is about 50-60, 50-55 or 55-60.

In some embodiments, the upper EAP layer (also referred to as the first EAP layer) has a Shore O durometer value of about 2-25. In some embodiments, the lower EAP layer (also referred to as the EAP second layer) has a Shore O durometer value of about 15-60.

In some embodiments, the lower EAP layer (also referred to as the second EAP layer) has a cross-link density of at least 1.5% vol/wt of cross-linking agent/linear monomers (e.g., with poly(ethylene glycol) dimethacrylate) and at least 6% vol/wt of cross-linking agent/linear monomers (e.g., with poly(dimethylsiloxane) dimethacrylate). In some embodiments, the upper EAP layer (also referred to as the first EAP layer) has a cross-link density of less than 1.5% vol/wt of cross-linking agent/linear monomers (e.g., with poly(ethylene glycol) dimethacrylate) and less than 6% vol/wt of cross-linking agent/linear monomers (e.g., with poly(dimethylsiloxane) dimethacrylate). In some embodiments, the lower EAP layer (also referred to as the second EAP layer) has a cross-link density of about 1.00%, 1.25%, 1.50%, 1.60%, 1.75%, 1.80%, 2.00%, 2.25%, or 2.50% higher for each cross-linking agent than that of the upper EAP layer (also referred to as the first EAP layer). In some embodiments, a small change in cross-link density causes a large change in the EAPs physical properties such as durometer.

In some embodiments, the first and/or second electroactive ionic polymers are selected from the group consisting of polymethacrylic acid, poly2-hydroxyethyl methacrylate, poly(vinyl alcohol), ionized poly(acrylamide), poly(acrylic acid), poly(acrylic acid)-co-poly(acrylamide), poly(2-acrylamide-2-methyl-1-propane sulfonic acid), poly(methacrylic acid), poly(styrene sulfonic acid), quarternized poly(4-vinyl pyridinium chloride), poly(vinylbenzyltrimethyl ammonium chloride), sulfonated poly(styrene-b-ethylene-co-butylene-b-styrene), sulfonated poly(styrene), and a combination thereof.

In some embodiments, the first and/or second electroactive ionic polymers are cross-linked with one or more cross-linking polymer agents each selected from the group consisting of a poly(dimethylsiloxane) (PDMS) dimethacrylate chain, a poly(ethylene glycol) dimethacrylate chain, an ethylene glycol dimethacrylate, 1,1,1-trimethylolpropane trimethacrylate, and a combination thereof. In some embodiments, the first and second electroactive polymers are cross-linked with different cross-linking agents and/or combinations of cross-lining agents so that the durometer value of the second electroactive ionic polymer is higher than that of the first electroactive ionic polymer.

In some specific embodiments, the first electroactive ionic polymer is cross-linked with one or more first cross-linking polymer agents which provides elasticity in the final EAP. Non-limiting examples of elastomeric causing cross-linking agents include poly(dimethylsiloxane) (PDMS) dimethacrylate. In some specific embodiments, the second electroactive ionic polymer is cross-linked with a second cross-linking polymeric agent selected from the group consisting of a poly(ethylene glycol) dimethacrylate chain, an ethylene glycol dimethacrylate, 1,1,1-trimethylolpropane trimethacrylate, and a combination thereof. In some specific embodiments, the second electroactive ionic polymer is cross-linked with poly(dimethylsiloxane) (PDMS) dimethacrylate and one or more other cross-linking agent selected from the group consisting of a poly(ethylene glycol) dimethacrylate chain, an ethylene glycol dimethacrylate, 1,1,1-trimethylolpropane trimethacrylate, and a combination thereof, such that the durometer value of the second electroactive ionic polymer is higher than that of the first electroactive ionic polymer.

In some embodiments, one or both of the lower and upper EAP layers are in electrical contact with a conductive layer electrically connected to an electrode (also referred to as the first electrode). In some embodiments, the conductive layer is made from a material selected from the group consisting of metal, carbon, and a combination thereof.

In some embodiments, the electroactive polymer-based system also includes a second electrode counter to the first electrode. In some embodiments, the first and/or second electrodes are flexible, bendable or stretchable electrodes. In some embodiments, the first and/or second electrodes are spiral-shaped or spring-shaped. In some embodiments, the first and/or second electrodes are made from a material selected from the group consisting of metal, carbon, other conductive materials, and a combination thereof. For simplicity, these elements, e.g., the first and second electrodes, conductive layers are not shown in FIGS. 1B and 2.

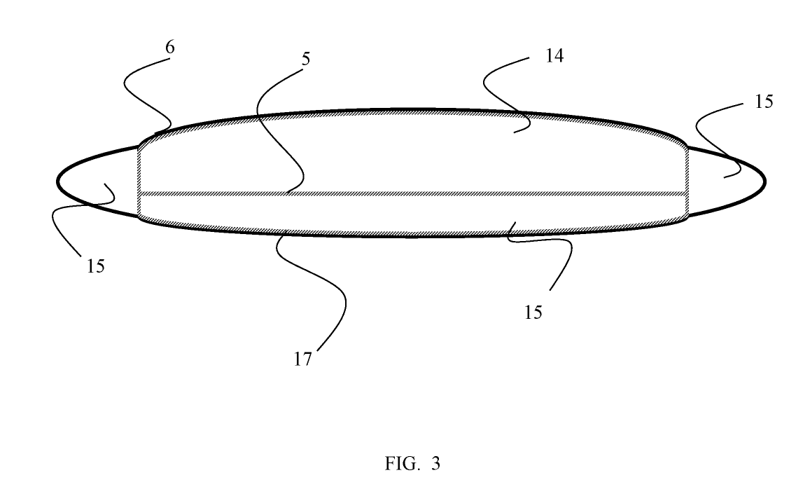

FIG. 3 shows the encapsulated EAP shape-morphing, sensing, and force attenuating system as a pad for the prosthetic liner or socket, where the lower (or outer) layer 15 of the pad comprises a firmer much less shape morphing (higher cross-link density) EAP zone, encapsulated in a flexible coating 6. The upper (or inner layer) of the pad 14 comprises a softer, much more shape-morphing (lower cross-link density) EAP zone. Alternatively, the lower layer 15 can be an open-cell foam reservoir. There can be additional open cell foam reservoirs for fluidic storage and fluidic flow. The two layers are separated by a conductive layer 5.

In another aspect, an electroactive polymer-based system is described, including:

one or more first electrodes;

a second electrode counter to the first electrode and spaced apart from the first electrode;

an ionically conductive fluid; and

an actuator electronically connected to the first electrodes and in fluidic communication with the second electrode, and comprising
an electroactive ionic polymer layer comprising an electroactive ionic polymer selected to expand or contract on application of an electrical potential; and
an array of a plurality of isolated conductive areas each in electric communication with an area of a plurality of areas of the electroactive ionic polymer layer; wherein the plurality of isolated conductive areas comprises at least one or more first isolated conductive areas in electric communication with the one or more first electrodes independent from other isolated conductive areas such that the areas of the electroactive ionic polymer layer in electric communication with the first isolated conductive areas are capable of being actuated independently.

In some embodiments, the electroactive polymer-based system further includes one or more third electrodes in electric communication with one or more second isolated conductive areas in the plurality of isolated conductive areas such that the areas of the electroactive ionic polymer layer in electric communication with the second isolated conductive areas are capable of being actuated independently from areas in the EAP layer in electric communication with the first isolated conductive areas.

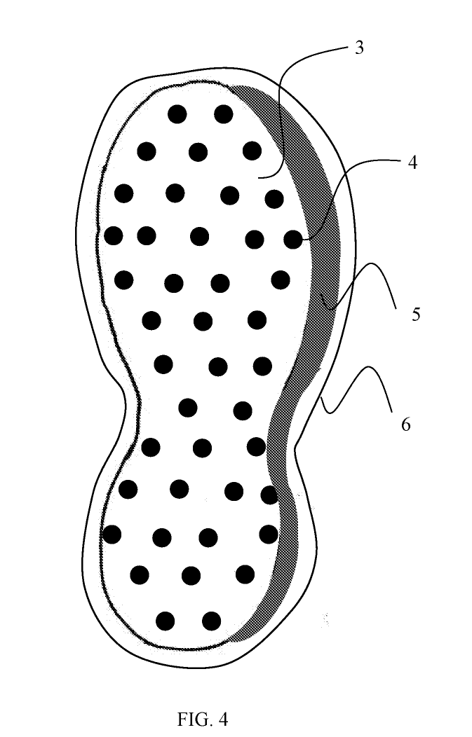

FIG. 4 shows an encapsulated EAP shape-morphing, sensing, and force attenuating system for shoe insoles, for example, with conductive layer 5 (which can be lined electrically to the first electrode) below the EAP layer 3, and a conductive layer above the EAP layer 3 and including an array of a plurality of isolated small (pixelated) conductive areas. This allows for shape-morphing in desired areas, by separately applying electric potential to one or more of the conductive areas. This also allows for sensing, from simple sensing, such as number of steps, to sophisticated pressure map sensing of the foot and foot strike during ambulation (walking and running) using pixilation of one or both conductive layers. The conductive layers can be carbon based particles, fibers, and/or weaves, or metal based particles, wires, or meshes, or a combination thereof. The EAPs being a non-Newtonian material (is a semi-solid hydrogel, with solid and liquid properties) also attenuates force, providing for a comfortable, healthy shoe insole.

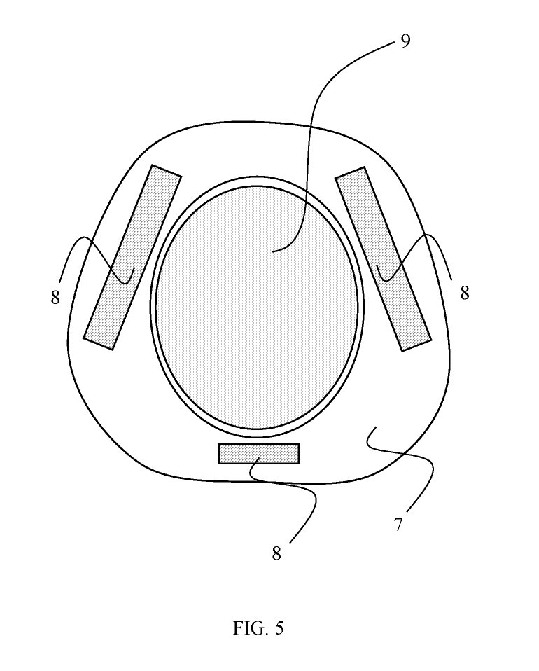

FIG. 5 shows an encapsulated EAP shape-morphing, sensing, and force attenuating system within a flexible prosthetic liner, where the EAP system shape-morphs for perfect fit, senses pressure to maintain perfect fit through an algorithm, and attenuates force, with good creep resistance and low hysteresis effects, for an extremely comfortable fit, particularly during ambulation (walking and running).

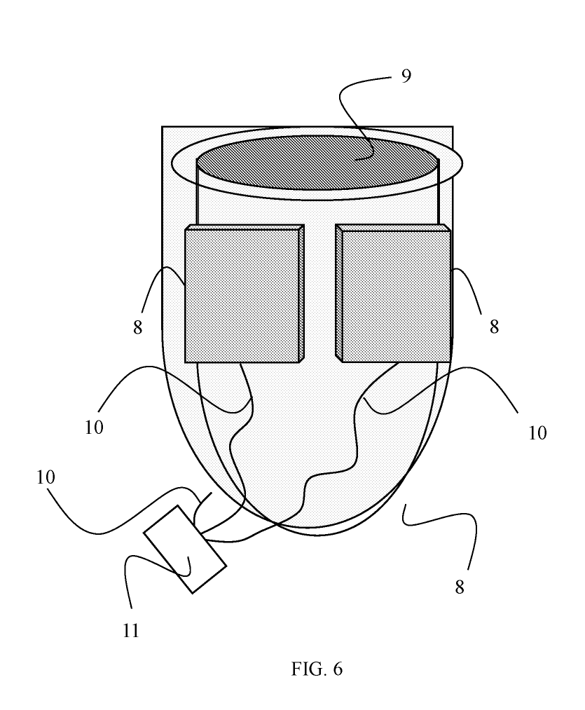

FIG. 6 shows the encapsulated EAP shape-morphing, sensing, and force attenuating system 8 within a flexible prosthetic liner, where the EAP system shape-morphs for perfect fit, senses pressure to maintain perfect fit through an algorithm, and attenuates force, with good creep resistance and low hysteresis effects, for an extremely comfortable fit, particularly during ambulation (walking and running) Also shown is a battery pack 11, which can have a three-way switch for EAP contraction, EAP expansion, and no electric input.

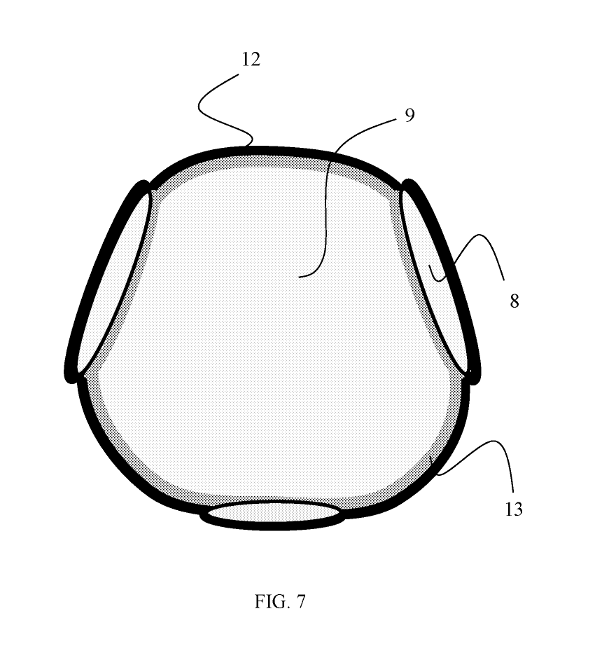

FIG. 7 is a cross-sectional of the encapsulated EAP shape-morphing, sensing, and force attenuating system 8 as pads in strategic locations within a prosthetic hard socket 12, where the EAP-based pads shape-morph for perfect fit, senses pressure to maintain perfect fit through an algorithm, and attenuates force, with good creep resistance and low hysteresis effects, for an extremely comfortable fit, particularly during ambulation (walking and running) Not shown are battery packs and switches, which can have a three-way switch for EAP contraction, EAP expansion, and no electric input.

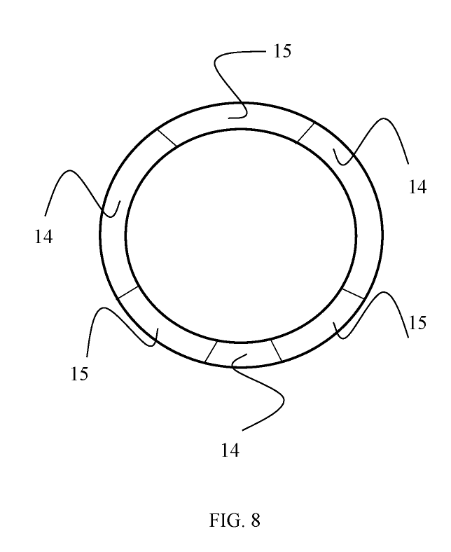

FIG. 8 shows an encapsulated EAP shape-morphing, sensing, and force attenuating system as a band in the prosthetic liner or prosthetic socket, where there are alternating zones within the band of more shape-morphing EAPs 14 (less cross-linked) and less shape-morphing EAPs 15 (more cross-linked) or open-cell foams. This can be expanded to describe an entire prosthetic liner comprising the EAP shape-morphing, sensing, and force attenuating materials, with different zones for different levels of desired shape-morphing abilities. Not shown is encapsulation, wiring, battery packs, and switches.

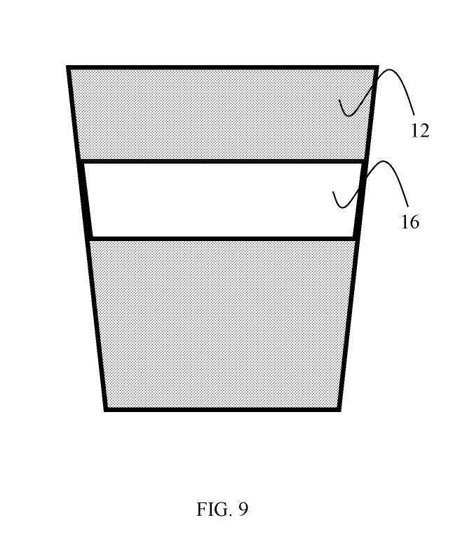

FIG. 9 shows the encapsulated EAP shape-morphing, sensing, and force attenuating system as an EAP band 16 in the prosthetic hard socket, located within the circumference of the prosthetic socket 12 to maintain fit around the residual limb. Not shown is wiring, battery packs, and switches.

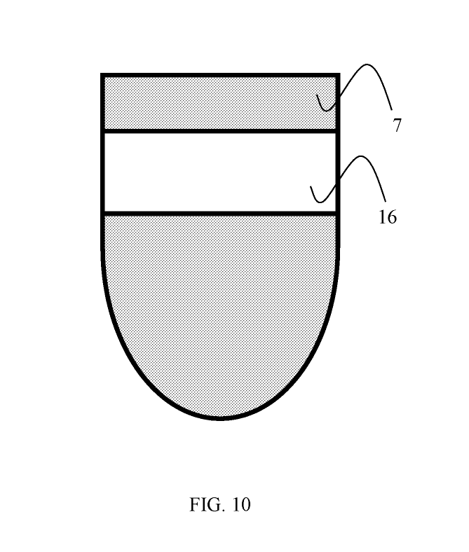

FIG. 10 shows the encapsulated EAP shape-morphing, sensing, and force attenuating system as an EAP band 16 in the prosthetic liner 7, located within the circumference of the prosthetic liner to maintain fit around the residual limb. Not shown is wiring, battery packs, and switches.

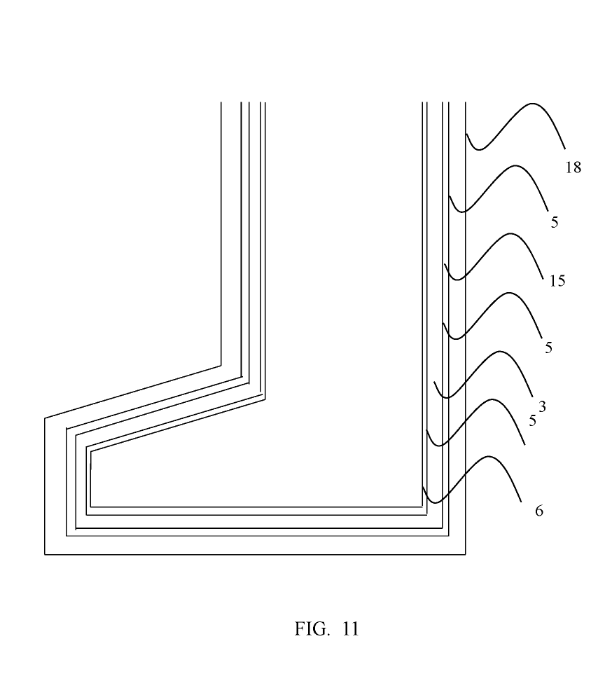

FIG. 11 shows the encapsulated EAP shape-morphing, sensing, and force attenuating system as a compression boot, where one or more layers of the EAP-based system comprising a conductive layer 5 and EAP layer 3, all encapsulated, compress the foot and/or leg. This can be programmed to compress the foot and/or leg in circumferential wave-like pattern(s), to massage and push excess fluid out of the limb towards the central body.

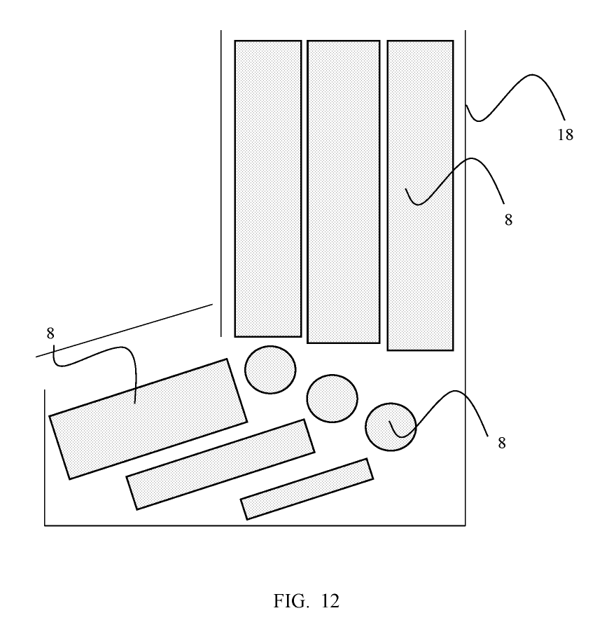

FIG. 12 shows the encapsulated EAP shape-morphing, sensing, and force attenuating system as a compression boot, in zones within the boot, including a plurality of EAP-based zones 8. Each of the EAP-based zone 8 may comprise a conductive layer and EAP layer, all encapsulated, compress the foot and/or leg. This can be programmed to compress the foot and/or leg in circumferential wave-like pattern(s), to massage and push excess fluid out of the limb towards the central body.

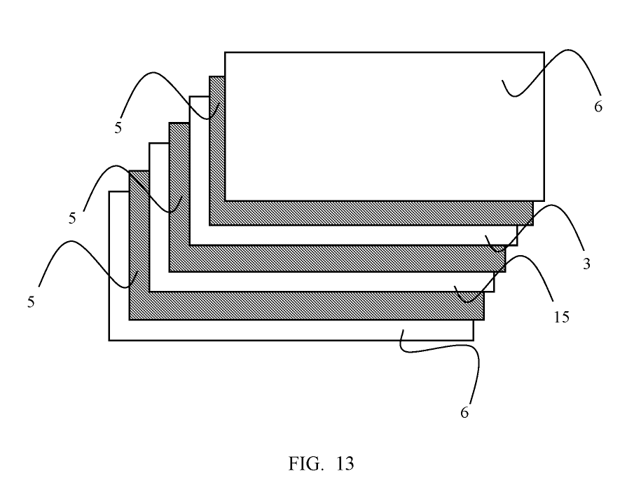

FIG. 13 shows the encapsulated EAP shape-morphing, sensing, and force attenuating system as compression tape, where one or more layers of the EAP-based system comprising a conductive layer 5 and EAP layer 3, all encapsulated, create a dynamic compressive tape or bandage. This can be programmed to compress the foot and/or leg in circumferential wave-like pattern(s), to massage and push excess fluid out of the limb towards the central body.

### LIST OF REFERENCE NUMERALS

1—electroactive polymer (EAP) layer of softer durometer
2—EAP layer of firmer durometer
3—actuator including one or more EAP shape-morphing, sensing, and force attenuating layer
4—small (pixel) conductive area
5—conductive layer
6—flexible coating (encapsulation)
7 surrounding flexible prosthetic liner
8—encapsulated EAP shape-morphing, sensing, and force attenuating system
9 human residual limb
10—flexible or bendable electrode(s)
11—battery pack
12—hard socket
13—standard flexible prosthetic liner
14—softer, more shape-morphing EAP zone
15—open cell foam or firmer, less shape-morphing EAP zone
16—EAP band
17—opposite charged conductive electrode layer
18—external hard component of compression boot
19—fluid reservoir containing electrolyte
20—first electrode
21—second electrode

Electroactive Polymers

In some embodiments, the first electroactive ionic polymer is cross-linked with a first cross-linking polymeric chain. In certain specific embodiments, the first electroactive ionic polymer is an elastomeric polymer chain. Non-limiting examples of the elastomeric polymer chains include a poly(dimethylsiloxane) (PDMS) chain, and a poly(dimethylsiloxane) (PDMS) dimethacrylate chain. In certain specific embodiments, the first electroactive ionic polymer is cross-linked with a first cross-linking polymeric agent comprising a poly(dimethylsiloxane) (PDMS) dimethacrylate chain and a second crossing-linking polymeric agent different from the first cross-linking polymeric agent. In some embodiments, the first electroactive ionic polymer is cross-linked with a first cross-linking polymeric chain comprising a poly(dimethylsiloxane) (PDMS) dimethacrylate chain and a second crossing-linking polymeric agent different from the first cross-linking polymeric agent. As described herein, the first electroactive ionic polymer may be cross-linked with a first cross-linking polymeric chain and a second crossing-linking polymeric agent different from the first cross-linking polymeric agent. In certain embodiments, the first cross-linking polymer agent has elastic characteristics. Non-limiting examples of the first cross-linking polymer agent include a poly(dimethylsiloxane) (PDMS) dimethacrylate polymeric chain. In certain embodiments, the second cross-linking polymeric agent is selected from the group consisting of a poly(ethylene glycol) dimethacrylate chain, an ethylene glycol dimethacrylate, 1,1,1-trimethylolpropane, and a combination thereof. In certain embodiments, the first electroactive ionic polymeric material is selected from the group consisting of polymers of methacrylic acid, copolymers of methacrylic acid and methacrylic acetate salt, such as potassium or sodium salt, other ion-containing polymers or copolymers, and combinations thereof.

Therefore, in these embodiments, the electroactive polymer may be multimodal. In these embodiments, the first electroactive ionic polymer may comprise two or more cross-linking polymeric agents and thus have more than one desirable property. In certain specific embodiments, the property is one or more characteristics selected from the group consisting of resistance, elasticity, firmness, shape-morphing ability, resiliency and a combination thereof. Further use of third and/or fourth cross-linking polymer agents different from the first and second cross-lining polymer agents is contemplated. That is, the electroactive polymer may further comprise a fourth cross-linking polymer agent different from the first, second, and third cross-linking polymer agents.

In some embodiments, the first and/or second electroactive ionic polymers are described. The first and/or second electroactive ionic polymers can be polymers of one or more ion-containing monomers or generally any polymer containing one or more ionizable groups. In certain embodiments, the first and/or second electroactive ionic polymers comprise ion-containing monomers such as methacrylic acid, which can also contain polymers comprising non-ionic monomers such as 2-hydroxyethyl methacrylate, cross-linked with poly(ethylene glycol) dimethacrylate or other suitable cross-linking agents, such as ethylene glycol dimethacrylate, 1,1,1-trimethylolpropane trimethacrylate, or a combination of cross-linking agents. Other electroactive polymers may also be used as the electroactive material or as a component of the electroactive material, such as poly(vinyl alcohol), ionized poly(acrylamide), poly(acrylic acid), poly(acrylic acid)-co-(poly(acrylamide), poly(2-acrylamide-2-methyl-1-propane sulfonic acid), poly(methacrylic acid), poly(styrene sulfonic acid), quartemized poly(4-vinyl pyridinium chloride), poly(vinylbenzyltrimethyl ammonium chloride), sulfonated poly(styrene-b-ethylene-co-butylene-b-styrene), sulfonated poly(styrene), or materials that respond to electricity by movement, expansion, contraction, curling, bending, buckling, or rippling. The preferred electroactive material comprises the monomer methacrylic acid, polymerized and cross-linked, preferably with the cross-linking agent poly(ethylene glycol) dimethacrylate with a number average molecular weight around 330 grams per mole, cross-linked at a low level, less than 0.78 mole percent poly(ethylene glycol) dimethacrylate with respect to methacrylic acid, preferably cross-linked within a range of 0.31 to 0.44 mole percent poly(ethylene glycol) dimethacrylate with respect to methacrylic acid. In certain embodiments, prior to polymerization, the monomer and cross-linking agent is diluted with a solvent miscible or compatible with the ion-containing monomer(s). Once polymerized and cross-linked, the electroactive material may be further swollen with an electrolyte solution or electrolyte gel formulation. Other suitable materials and compositions for the electroactive material are described in U.S. Pat. Nos. 8,088,453, 7,935,743, and 5,736,590 and U.S. Ser. Nos. 13/843,959 and 14/476,646, the contents of which are expressly incorporated by reference.

In certain embodiments, different formulations, preferably with respect to cross-linking formulations containing electroactive polymers with different levels of cross-linking, can be used in different regions of the polymer in the prosthetic liner or other actuating or void-filling system to provide for different levels of softness, hardness, or shape-morphing as needed. In certain embodiments, multiple cross-linking strategies can be used to provide for multi-modality and impact resistance over a wide range of impact scenarios, and to be able to withstand repeated impacts from typical use.

In yet another aspect, an actuation device comprising one or more of the shape-morphing systems disclosed herein is described, wherein upon the application of an electrical potential to the first electrode, the first electroactive ionic polymer is configured to expand or contract to generate an actuation force to result in a movement of at least a portion of the actuation device from a first position to a second position.

In some embodiments, the electroactive polymer-based system also includes an electroconductivity-enhancing material in ionic communication with the first and/or electroactive ionic polymer. In some embodiments, the electroconductivity-enhancing material is selected from the group consisting of solvent, electrolyte solution, electrolyte gel formulation, carbon particles, conductive fibers, preceding weaves, preceding felts, preceding nano-particles, preceding nanotubes, metal ions, salt, and a combination thereof. In some embodiments, the electrolytes in the EAPs and EAP-based systems can be of the group comprising Group 1 ions and Group 7 ions, the group comprising Group 1 ions and sulfate or other anionic counter ions, the group comprising Group 2 ions and sulfate or other anionic counter ions, and combinations thereof. Non-Group 7 anions in the electrolyte solution component of the EAPs have the advantage of releasing oxygen gas when these EAPs are electrically activated and above the electrophoresis threshold of 1.23 V. Small amounts of oxygen expression in the prosthetic liner or hard socket can be therapeutic to the skin of residual limbs, and very therapeutic to the skin of the foot and/or leg being treated in the compression boot. Standard fuel cells require hydrogen and oxygen, releasing water and providing electricity. The EAPs and EAP-based systems in the instant invention operate best moist, so require water, and actuate with electric input.

In some embodiments, the electroactive polymer-based system further includes a power source. In some embodiments, the power source is a rechargeable or non-rechargeable battery pack. In some embodiments, above the electrophoresis threshold of 1.23 V, these EAPs may release hydrogen and oxygen. EAP actuation above the electrophoresis threshold of 1.23 V can allow for tie-in with a fuel cell(s), for energy efficient actuation of the EAPs and EAP-based systems in the instant invention.

In some embodiments, the electroactive polymer-based system is in a form selected from the group consisting of fibers, bulk, slabs, bundles, and combinations thereof.

### FURTHER DESCRIPTION OF EMBODIMENT(S)

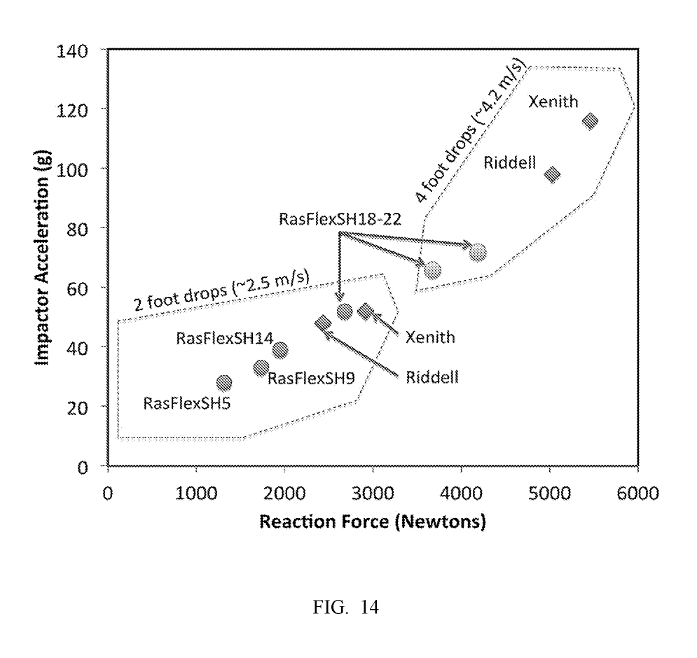

The EAPs and EAP-based systems in the instant invention are ideally suited for impact attenuation since these EAPs are neither a pure solid nor pure liquid. Due to the material's semi-solid composition and viscoelastic and damping properties, these EAPs and EAP-based systems exhibit non-Newtonian behavior. FIG. 14 illustrates drop tower data comparing EAPs in the instant invention (EAP samples in the instant invention are the RasFlex series) to traditional padding in Xenith and Riddell football helmets. Note the superior performance data of RasFlexSH18-22 from the high impact 4-foot impactor drops. From impact testing, in addition to the attenuation of impact force directly through the material (FIG. 14), the 20×20×2.5 cm sample also propagated the force laterally with distinct wave-like behavior, which was observed using high-speed photography. The architecture of helmet padding using the EAPs and EAP-based systems in the instant invention can also come into play, where different layers or areas of the EAPs and EAP-based systems in the instant invention may be able to turn direct impact into glancing impact within the same EAP-based system (FIG. 1). Shaped and layered approaches, including anisotropy, potentially provide the EAPs and EAP-based systems with the ability to both attenuate the impact force through the padding and concurrently mitigate or spread the impact force laterally. Nano-level considerations in design and production of these EAPs and EAP-based systems can improve the speed of electro-actuation for potential shape-morphing in response to incoming impacts.

In an iterative design-test-redesign-retest cycle, EAP samples in the instant invention (RasFlex series in FIG. 14) of varying stiffness were developed, and exposed to drop tower impacts at approximately 2.5 or 4.2 m/s. The impactor consisted of a 5 kg carriage with cylindrical impact face that was dropped from a height onto a rigidly-held padding specimen. For comparison, the aforementioned test procedure was performed on football helmet padding from off-the-shelf helmets (Riddell and Xenith), which were selected because of their use by the NFL and because of their high ranking in helmet testing (Virginia Tech, http://www.beam.vt.edu/helmet/helmets_football.php). Note that the RasFlexSH18-22 EAP formulation provides impact attenuation that is comparable to the Riddell and Xenith padding at 2 foot drop (˜2.5 m/s), and provides better attenuation at drops from 4 feet (˜4.2 m/s). Drop testing was also conducted on samples previously frozen to −79° C. and then thawed to room temperature, on samples at 0° C., on samples at 40° C., and on samples heated to 100° C. and then returned to room temperature. No change in drop test performance or damage to material structure was found. An EAP RasFlex sample was also exposed to repeated low level impacts (1200 impacts at 908 N, 0.1s duration triangular wave, with 30 s wait time between impacts) and detected no damage to the material and no difference between pre- and post-drop testing at 2.4 and 4.5 m/s. In addition, the EAP and EAP systems in the instant invention operate well within the relative humidity range of 40% to 100%, and even uncoated, Ras Labs smart materials operate well in water, including the salinity of ocean water. For use as padding or liners, these EAPs and EAP-based systems are coated, such as with medical grade silicone.

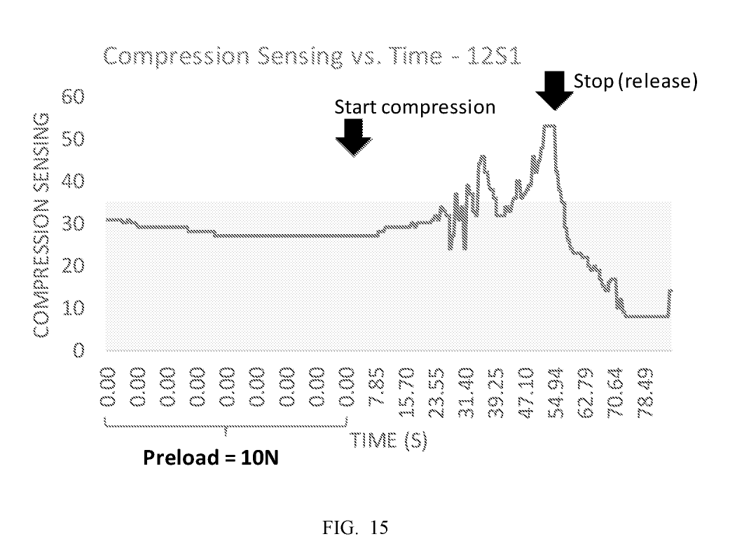

The EAPs and EAP-based systems in the instant invention are variable resistors, and can sense mechanical pressure, from high impact (FIG. 14) to gentle pressure (FIG. 15). FIG. 15 shows the sensing abilities of these EAP and EAP-based systems under much gentler mechanical pressure than FIG. 14. The compression sensing of EAP Sample LA_12 was determined using an Instron® Model 4466 Universal Testing Machine, at a speed of 3 mm/min with a peak compressive force of 174.964 N. Once the plates reached close to touching, the automatic stop provided an immediate release of pressure, which was also observed (FIG. 15). To track the pressure on the EAP, conductive layers were attached above and below the EAP, with wiring attached to an Arduino® micro-processor, which was connected to a laptop computer for real-time analysis and data capture during the compression testing. For both prosthetic and robotics, what is missing is a convenient, streamlined system to provide sensory feedback, such a mechanical pressure—what we know as touch. The EAPs and EAPs in the instant invention provide for shape-morphing, good sensing abilities (gentle touch to high impact), and force attenuation.

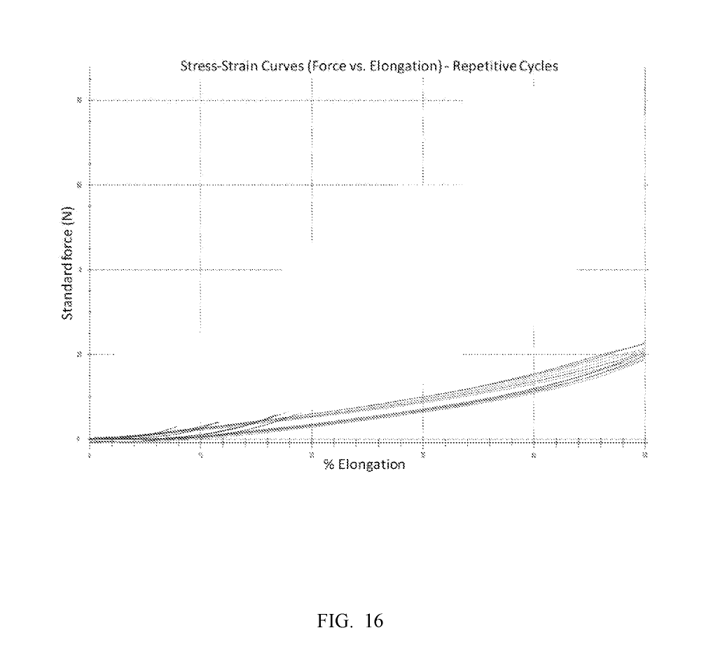

In addition to the shape-morphing, sensing, and force attenuation abilities, the EAPs and EAPs in the instant invention also provide for good creep resistance, good elasticity, and low hysteresis effects (FIG. 16). The creep resistance and hysteresis effects are magnitudes better than standard liner materials currently in the marketplace. This attribute in these EAP and EAP-based systems provide for very good elastic rebound of the material in the prosthetic liner (or as the prosthetic liner) and in pads for the hard socket, which will provide for improved comfort for the amputee, particularly during ambulation (walking and running).

This EAP-based thin film can be applied to many other applications, such as a sensing “skin” for pressure feedback (touch) on a robotic or prosthetic arm, for example. These EAPs and EAP-based systems in the instant invention could also be used as sensing pads, such as “fingertips” on a robotic or prosthetic hand, for example. Robotic hands are also known as grippers or end effectors. As a covering for a prosthetic or robotic arm or hand, in addition to sensing pressure, like skin, the covering could also be shape-morphing, with underlying areas attached to the linkages making the hand or arm, so in addition to sensing and looking life-like, the EAP-based covering could also assist with desired movement rather than being passive weight, thus act as muscles, alone or acting in combination (hybrid approach) with traditional prosthetic and robotic hands and arms.

1. An electroactive polymer-based system, comprising:
a first electrode;
a second electrode counter to the first electrode and spaced apart from the first electrode;
an ionically conductive fluid; and
an actuator electronically connected to the first electrode and in fluidic communication with the second electrode, and comprising
a first electroactive ionic polymer layer comprising a first electroactive ionic polymer; and
a second electroactive ionic polymer layer comprising a second electroactive ionic polymer; wherein the first and second electroactive polymers are selected to expand or contract on application of an electrical potential; wherein the Shore O durometer value of the second electroactive ionic polymer is higher than that of the first electroactive ionic polymer; and wherein the first and second electroactive ionic polymer layers are configured to transfer the force applied onto the first electroactive ionic polymer layer to the second electroactive ionic polymer layer to be attenuated.

2. The electroactive polymer-based system of claim 1, wherein the first and second electroactive ionic polymer layers are in direct contact with each other or in close proximity to each other.

3. The electroactive polymer-based system of claim 1, wherein the first and second electroactive ionic polymer layers are separated by a soft or elastic layer.

4. The electroactive polymer-based system of claim 1, wherein the difference of Shore O durometer values between the first and second electroactive ionic polymer layers is about 2-60.

5. The electroactive polymer-based system of claim 1, wherein the first electroactive ionic polymer has a cross-link density of at least about 1.5%-6.0% vol/wt of cross-linking agent/linear monomers.

6. The electroactive polymer-based system of claim 1, wherein the second electroactive ionic polymer has a cross-link density of less than about 1.5% vol/wt of cross-linking agent/linear monomers.

7. The electroactive polymer-based system of claim 1, wherein the second electroactive ionic polymer has a cross-link density of about 0.50%, 0.75%, 1.00%, 1.25%, 1.50%, 1.60%, 1.75%, 1.80%, 2.00%, 2.25%, or 2.50% higher for each cross-linking agent than that of the first electroactive ionic polymer.

8. The electroactive polymer-based system of claim 1, wherein the second electroactive ionic polymer layer has a shape selected from the group consisting of conical, half-ovoid, ovoid, sheet, pad, sphere, cylinder, cone, pyramid, prism, spheroid ellipse, ellipsoid, rectangular prism, toroid, parallelepiped, rhombic prism shapes and a combination thereof.

9. The electroactive polymer-based system of claim 1, wherein the second electroactive ionic polymer layer has a shape selected from the group consisting of a conical shape, a half-ovoid shape, an ovoid shape, and a combination thereof.

10. The electroactive polymer-based system of claim 1, wherein the first electroactive ionic polymer layer has a shape reciprocal to the shape of the second electroactive ionic polymer layer.

11. The electroactive polymer-based system of claim 1, further comprising one or more electrically conducting layers in electrical contact with the actuator and the first electrode.

12. The electroactive polymer-based system of claim 11, wherein the electroactive polymer-based system comprises a first and second electrically conducting layers in electrical contact with the first and second electroactive ionic polymer layers, respectively.

13. The electroactive polymer-based system of claim 11, wherein the electrically conducting layer comprises an array of a plurality of electrically conducting areas.

14. The electroactive polymer-based system of claim 1, further comprising a fluid reservoir in fluidic communication with the first and second electroactive ionic polymers and connected to the second electrode.

15. The electroactive polymer-based system of claim 14, wherein the fluid reservoir is in the second electroactive ionic polymer layer.

16. The electroactive polymer-based system of claim 1, wherein the first and/or second electroactive ionic polymers are each independently selected from the group consisting of polymethacrylic acid, poly-2-hydroxyethyl methacrylate, poly(vinyl alcohol), ionized poly(acrylamide), poly(acrylic acid), poly(acrylic acid)-co-(poly(acrylamide), poly(2-acrylamide-2-methyl-1-propane sulfonic acid), poly(methacrylic acid), poly(styrene sulfonic acid), quarternized poly(4-vinyl pyridinium chloride), poly(vinylbenzyltrimethyl ammonium chloride), sulfonated poly(styrene-b-ethylene-co-butylene-b-styrene), sulfonated poly(styrene), and a combination thereof.

17. The electroactive polymer-based system of claim 1, wherein the first and/or second electroactive ionic polymers are cross-linked with one or more cross-linking polymer agents each selected from the group consisting of a poly(dimethylsiloxane) (PDMS) dimethacrylate chain, a poly(ethylene glycol) dimethacrylate chain, an ethylene glycol dimethacrylate, 1,1,1-trimethylolpropane trimethacrylate, and a combination thereof.

18. The electroactive polymer-based system of claim 1, wherein the first electroactive ionic polymer is cross-linked with one or more first cross-linking polymer agents which is elastomeric or provides elasticity.

19. The electroactive polymer-based system of claim 18, wherein the first cross-linking polymeric agent comprising a poly(dimethylsiloxane) (PDMS) dimethacrylate chain.

20. The electroactive polymer-based system of claim 18, wherein the second electroactive ionic polymer is cross-linked with a second cross-linking polymeric agent selected from the group consisting of a poly(dimethylsiloxane) dimethacrylate, a poly(ethylene glycol) dimethacrylate chain, an ethylene glycol dimethacrylate, 1,1,1-trimethylolpropane trimethacrylate, and a combination thereof; wherein the second electroactive ionic polymer is cross-linked at a higher level than that of the first electroactive ionic polymer.

21. An electroactive polymer-based system, comprising:
one or more first electrodes;
a second electrode counter to the first electrode and spaced apart from the first electrode;
an ionically conductive fluid; and
an actuator electronically connected to the first electrodes and in fluidic communication with the second electrode, and comprising
an electroactive ionic polymer layer comprising an electroactive ionic polymer selected to expand or contract on application of an electrical potential; and
an array of a plurality of isolated conductive areas each in electric communication with a plurality of areas of the electroactive ionic polymer layer; wherein the plurality of isolated conductive areas comprises at least one or more first isolated conductive areas in electric communication with the one or more first electrodes independent from other isolated conductive areas such that the areas of the electroactive ionic polymer layer in electric communication with the first isolated conductive areas are capable of being actuated independently.

22. The electroactive polymer-based system of claim 21, wherein the electroactive polymer-based system further includes one or more third electrodes; the plurality of isolated conductive areas comprises at least one or more second isolated conductive areas in electric communication with the one or more third electrodes independent from other isolated conductive areas such that the areas of the electroactive ionic polymer layer in electric communication with the second isolated conductive areas are capable of being actuated independently.

23. A liner for securing a limb in a prosthetic device or a prosthetic socket comprising:
a flexible layer configured to surround a limb of a patient or conform to the inside circumference of a prosthesis; and
at least one electroactive polymer-based system of claim 1 or 21 embedded in the flexible layer and configured to secure or engage a limb of a patient.

24. The liner or prosthetic socket of claim 23, wherein the flexible layer is made of silicone.

25. The liner or prosthetic socket of claim 23, wherein the liner or prosthetic socket comprises a plurality of the electroactive polymer-based system of any one of the preceding claims and embedded in the flexible layer; wherein the electroactive polymer-based systems are fluidically isolated from each other and arranged around the limb of a patient to secure the limb.

26. The liner or prosthetic socket of claim 23, wherein the prosthesis has a hard body and upon the application of an electrical potential to the first electrode, the actuator is configured to expand against the hard body towards the limb of the patient.

27. A shoe insole comprising an electroactive polymer-based system of claim 1 or 21.

28. A protective gear comprising an electroactive polymer-based system of claim 1 or 21.

29. The protective gear of claim 28, wherein the protective gear is a helmet.

30. A compression equipment comprising an electroactive polymer-based system of claim 1 or 21.

31. The compression equipment of claim 30, wherein the compression equipment is a compression boot for diabetic patients, a military anti-shock trouser (MAST, also called pneumatic anti-shock garments (PASG)) for trauma patients, a compression bandage, a compression tape, or a compressive therapy.
